(19) United States
(12) Patent Application Publication
(10) Pub. No.: US 2004/0132024 A1
(43) Pub. Date: Jul. 8, 2004

(54) Method of detecting genetic disorders

(76) Inventors: Lawrence Shin Hsin Wu, Taipei (TW); Lichih Huang, Chiai (TW); Cherry Kuan-Ju Lin, Taipei (TW); Julia Kuei-Ting Yu, Nantou City (TW); Jui-Lin Chen, Taipei (TW); Jiu-Yao Wang, Tainan (TW)

Correspondence Address: FISH & RICHARDSON PC, 225 FRANKLIN ST, BOSTON, MA, 02110, US

(21) Appl. No.: 10/338,321

(22) Filed: Jan. 8, 2003

(51) Int. Cl. C12Q 1/68

(52) U.S. Cl. 435/6

(57) ABSTRACT

A method of developing a means for detecting a genetic disorder. The method involves identifying a microsatellite marker and a single nucleotide polymorphism marker. Presence of both the microsatellite marker and the single nucleotide polymorphism marker in a subject indicates that the subject is suffering from or at risk for suffering from a genetic disorder.

### BACKGROUND

Immunoglobulin E (IgE) plays an important role in development of allergic disorders including asthma. High serum IgE levels correlate with clinical expression of allergy. See Johansson et al. (1972) Prog. Clin. Immunol. 1:157-181, Burrows et al. (1989) N. Engl. J. Med. 320:271-277, Sears et al. (1991) N. Engl. J. Med. 325:1067-1071, and Halonen et al. (1992) Am. Rev. Respir. Dis. 146:866-870. Epidemiologic studies have shown that high IgE levels are associated with bronchial hyperresponsiveness, a major component of the asthma phenotype. See Sears et al. (1991) N. Engl. J. Med. 325:1067-1071, Hopp et al. (1984) J. Allergy Clin. Immunol. 73(2):154-158, and Burrows et al. (1991) J. Allergy Clin. Immunol. 88:870-877.

### SUMMARY

The present invention relates to a method of diagnosing a genetic disorder based on presence of both a microsatellite marker and a single nucleotide polymorphism marker in a subject.

In one aspect, the invention features a method of developing a means for detecting a genetic disorder in a subject. The method involves identifying a microsatellite marker and a single nucleotide polymorphism marker. Presence of both the microsatellite marker and the single nucleotide polymorphism marker in a subject indicates that the subject is suffering from or at risk for suffering from a genetic disorder. Such microsatellite and single nucleotide polymorphism markers can be identified based on their physical association (e.g., on the same chromosome or near each other) or functional relevance (e.g., both related to a particular disease). For example, D5S2011 and CD14-F1826 have been found to be associated with high IgE asthma based on the fact that they are close to each other on chromosome 5. Also within the scope of the invention is a kit for detecting a genetic disorder in a subject. The kit contains a first agent for detecting a microsatellite marker and a second agent for detecting a single nucleotide polymorphism marker. Detection of both the microsatellite and single nucleotide polymorphism markers in a subject indicates that the subject is suffering from or at risk for suffering from a genetic disorder.

In another aspect, this invention features a method of determining whether a subject is suffering from or at risk for suffering from a genetic disorder. The method involves providing a nucleic acid sample from a subject and detecting a microsatellite marker and a single nucleotide polymorphism marker. Detection of both the microsatellite marker and the single nucleotide polymorphism marker indicates that the subject is suffering from or at risk for developing a genetic disorder.

In one example, the microsatellite marker is a D5S2011 E marker and the single nucleotide polymorphism marker is a CD14-F1826 T marker. Detection of both the D5S2011 E marker and the CD14-F1826 T marker indicates that the subject is suffering from or at risk for suffering from an allergic disorder and having or at risk for having a serum IgE concentration of more than 200 IU/ml (e.g., 1000 IU/ml or higher). D5S2011 is a human di-nucleotide repeat microsatellite marker located at 5q31. A subject having a D5S2011 E marker can have either an EE or EX genotype. CD14-F1826 is a human single nucleotide polymorphism marker (T/C) located in the enhancer region of the CD14 gene (position −2984) on chromosome 5. A subject having a CD14-F1826 T marker can have either a TT or TC genotype. Allergic disorders include asthma and non-asthmatic atopy (e.g., atopic dermatitis, type-I diabetes, osteoporosis, inflammatory bowel disease, and allergic rhinitis). A subject to be diagnosed can be an individual from a Mongoloid population such as a Taiwanese population. The invention also provides a kit for detecting an allergic disorder. The kit contains a first agent for detecting a D5S2011 E microsatellite marker and a second agent for detecting a CD14-F1826 T single nucleotide polymorphism marker.

Furthermore, the present invention features a method of subtyping an allergic disorder. In one example, the method involves providing a nucleic acid sample from a subject suffering from or being at risk for suffering from an allergic disorder and detecting a D5S2011 E microsatellite marker. Detection of the D5S2011 E marker indicates that the subject is having or at risk for having a serum IgE concentration of more than 200 IU/ml (e.g., 1000 IU/ml or higher). The invention also provides a packaged product that includes (1) a container, (2) an agent for detecting a D5S2011 E microsatellite marker, and (3) a legend associated with the container and indicating that presence of the D5S2011 E marker in a subject indicates that the subject is suffering from or at risk for suffering from an allergic disorder and having or at risk for having a serum IgE concentration of more than 200 IU/ml (e.g., 1000 IU/ml or higher).

In another example, the method involves providing a nucleic acid sample from a subject suffering from or being at risk for suffering from an allergic disorder and detecting a D5S2011 J microsatellite marker. Detection of the D5S2011 J marker indicates that the subject is having or at risk for having a serum IgE concentration of less than 1000 IU/ml (e.g., 200 IU/ml or lower). The invention also provides a packaged product that includes (1) a container, (2) an agent for detecting a D5S2011 J microsatellite marker, and (3) a legend associated with the container and indicating that presence of the D5S2011 J marker in a subject indicates that the subject is suffering from or at risk for suffering from an allergic disorder and having or at risk for having a serum IgE concentration of less than 1000 IU/ml (e.g., 200 IU/ml or lower).

In still another example, the method involves providing a nucleic acid sample from a subject suffering from or being at risk for suffering from an allergic disorder and detecting a CD14-F1826 T single nucleotide polymorphism marker. Detection of the CD14-F1826 T marker indicates that the subject is having or at risk for having a serum IgE concentration of more than 200 IU/ml (e.g., 1000 IU/ml or higher). The invention also provides a packaged product that includes (1) a container, (2) an agent for detecting a CD14-F1826 T single nucleotide polymorphism marker, and (3) a legend associated with the container and indicating that presence of the CD14-F1826 T marker in a subject indicates that the subject is suffering from or at risk for suffering from an allergic disorder and having or at risk for having a serum IgE concentration of more than 200 IU/ml (e.g., 1000 IU/ml or higher).

The methods, kits, and packaged products of this invention are useful for diagnosing, preventing, and treating genetic disorders. The details of one or more embodiments of the invention are set forth in the accompanying description below. Other advantages, features, and objects of the invention will be apparent from the detailed description, and from the claims.

### DETAILED DESCRIPTION

The present invention is based on an unexpected discovery that concurrent presence of a microsatellite marker and a single nucleotide polymorphism marker in a subject indicates a genetic disorder more reliably than presence of each individual marker. As demonstrated in the example below, asthma patients having a D5S2011 E microsatellite marker are about 3 times more likely to have a serum IgE concentration of 1000 IU/ml or higher than those without the D5S2011 E marker. On the other hand, asthma patients having a CD14-F1826 T single nucleotide polymorphism marker are more than 2 times more likely to have a serum IgE concentration of 1000 IU/ml or higher than those without the CD 14-F1826 T marker. Surprisingly, asthma patients having both the D5S2011 E marker and the CD14-F1826 T marker are more than 5 times more likely to have a serum IgE concentration of 1000 IU/ml or higher than those having neither of the two markers. Accordingly, this invention features a method of developing a means for detecting a genetic disorder by identifying a microsatellite marker and a single nucleotide polymorphism marker in a subject. Presence of both markers is indicative of a genetic disorder.

Microsatellites are short tandem repeats (STRs) of 2˜6 bps which are widely dispersed throughout the human genome (Amos and Rubinsztein (1996) Nature Genetics 12:13-14 and Edwards et al. (1991) Am. J. Hum. Genet. 49:746-756). They have been extensively used for linkage mapping as well as forensic and population studies (Bowcock et al. (1994) Nature 368:455-457 and Brinkmann et al. (1996) Hum. Genet. 98:60-64). Polymorphism observed at these loci is due to variation in the number of repeats of a single unit (Valdes et al. (1993) Genetics 133:737-749 and Levinson and Gutman (1987) Mol. Biol. Evol. 4:203-221).

A microsatellite marker can be detected by obtaining nucleic acid from a subject, amplifying a segment of the nucleic acid with a pair of primers, and identifying the amplified segment. Primer sequences can be either retrieved from public databases or designed by a software program based on oligonucleotide properties such as annealing temperature and internal pairing. Nucleic acid can be prepared from a tissue sample or a fluid sample according to methods well known in the art. Amplification of the nucleic acid, e.g., by polymerase chain reaction (PCR), can be carried out following standard procedures. See, e.g., Ausubel et al. (1989) Current Protocols in Molecular Biology, John Wiley and Sons, New York; and Innis et al. (1990) PCR Protocols: A Guide to Methods and Applications. Academic Press, Harcourt Brace Javanovich, New York.

Identification of amplified segments of different sizes may be achieved using standard methods such as size fractionation, mass spectrometry-based detection, or any other fragment sizing technologies. Size fractionation separates DNA molecules according to their sizes, e.g., polyacrylamide gel electrophoresis. Size fractionation may also be accomplished by chromatographic methods known as gel filtration. The DNA segments in solution are separated according to their sizes as they pass through a column packed with a chromatographic gel. Mass spectrometry provides a means of “weighing” a DNA molecule by ionizing the molecule in vacuum and making it “fly” by volatilization. It can be used to simultaneously identify many DNA molecules. See, e.g., U.S. Pat. No. 6,268,144.

To facilitate the identification of amplified segments of different sizes, amplified segments can be labeled either during amplification, e.g., by the incorporation of labeled nucleotides, or using labeled primers. In addition to radioactive labels, other labels such as fluorescence, chemiluminescence, and electrochemical luminescence can be used. See Kricka (1992) Nonisotopic DNA Probe Techniques Academic Press, San Diego, pp. 3-28. Examples of fluorescent labels include fluoresceins, rhodamines (U.S. Pat. Nos. 5,366,860, 5,936,087, and 6,051,719), cyanines (U.S. Pat. No. 6,080,868 and WO 97/45539), and metal porphyrin complexes (WO 88/04777). In particular, fluorescence can be 6-carboxyfluorescein (FAM), 2′,4′,1,4,-tetrachlorofluorescein (TET), 2′,4′,5′,7′,1,4-hexachlorofluorescein (HEX; U.S. Pat. No. 5,654,442), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (U.S. Pat. Nos. 5,188,934 and 5,885,778), or 2′-chloro-7′-phenyl-1,4-dichloro-6-carboxyfluorescein 6 (U.S. Pat. No. 6,008,379). Rhodamine can be tetramethyl-6-carboxyrhodamine (TAMRA) or tetrapropano-6-carboxyrhodamine (ROX), and cyanine can be anthraquinone, malachite green, or a nitrothiazole or nitroimidazole compound.

Labeled amplified segments can be characterized directly by autoradiography or by laser detection, followed by computer assisted graphic display and analysis. For example, when different fluorescent labels are used, multiplexed or pooled PCR products can be analyzed simultaneously by using CCD camera, Genescan, and Genotyper softwares (Applied Biosystems). Genescan and Genotyper softwares can further manipulate the data by automatically inputting marker names from a data file and outputting them into data format of Excel or Text.

A single nucleotide polymorphism (SNP) occurs at a polymorphic site occupied by a single nucleotide, which is the site of variation between allelic sequences. “Polymorphic” refers to the occurrence of two or more genetically determined alternative sequences or alleles in a population of subjects. An SNP usually arises due to substitution, e.g., a transition or transversion, of one nucleotide for another at the polymorphic site. A transition is the replacement of one purine by another purine or one pyrimidine by another pyrimidine. A transversion is the replacement of a purine by a pyrimidine or vice versa. SNPs can also arise from a deletion of a nucleotide or an insertion of a nucleotide relative to a reference allele.

There are a variety of suitable procedures known in the art that can be employed to detect an SNP marker. Some of them are described in further detail below.

(1) Allele-Specific Probes

The design and use of allele-specific probes for analyzing polymorphisms is known in the art (see, e.g., EP 235,726 and WO 89/11548). Allele-specific probes can be designed to hybridize differentially, e.g., to hybridize to a segment of DNA from one individual but not to a corresponding segment from another individual, based on the presence of polymorphic forms of the segment. Relatively stringent hybridization conditions can be utilized to cause a significant difference in hybridization intensity between alleles, and possibly to obtain a condition wherein a probe hybridizes to only one of the alleles. Probes can be designed to hybridize to a segment of DNA such that the polymorphic site aligns with a central position of the probe.

Allele-specific probes can be used in pairs, wherein one member of the pair matches perfectly to a reference form of a target sequence, and the other member of the pair matches perfectly to a variant of the target sequence. The use of several pairs of probes immobilized on the same support may allow simultaneous analysis of multiple polymorphisms within the same target sequence.

(2) Tiling Arrays

Polymorphisms can also be identified by hybridization to nucleic acid arrays (see, e.g., WO 95/11995). WO 95/11995 also describes subarrays that are optimized for detection of a variant form of a precharacterized polymorphism. Such a subarray contains probes designed to be complementary to a second reference sequence, which is an allelic variant of the first reference sequence. The second group of probes is designed to exhibit complementarily to the second reference sequence. The inclusion of a second group (or further groups) can be particular useful for analyzing short subsequences of the primary reference sequence in which multiple mutations are expected to occur within a short distance commensurate with the length of the probes (i.e., two or more mutations within 9 to 21 bases).

(3) Allele-Specific Primers

An allele-specific primer hybridizes to a site on target DNA overlapping a polymorphism and only primes amplification of an allelic form to which the primer exhibits perfect complementarily. See, e.g., Gibbs (1989) Nucleic Acid Res. 17:2427-2448. Such a primer can be used in conjunction with a second primer which hybridizes at a distal site. Amplification proceeds from the two primers, leading to a detectable product signifying that the particular allelic form is present. A control is usually performed with a second pair of primers, one of which shows a single base mismatch at the polymorphic site and the other of which exhibits perfect complementarily to a distal site. The single-base mismatch prevents amplification and no detectable product is formed. The method can be optimized by including the mismatch in the 3′-most position of the oligonucleotide aligned with the polymorphism because this position is most destabilizing to elongation from the primer. See, e.g., WO 93/22456.

(4) Direct Sequencing

The direct analysis of the sequence of polymorphisms of the present invention can be accomplished using either the dideoxy chain termination method or the Maxam Gilbert method (see Sambrook et al. (1989) Molecular Cloning, A Laboratory Manual, 2nd Ed., CSHP, New York and Zyskind et al. (1988) Recombinant DNA Laboratory Manual, Acad. Press).

(5) Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. See Erlich ed. (1992) PCR Technology, Chapter 7: Principles and Applications for DNA Amplification, W. H. Freeman and Co, New York.

(6) Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al. (1989) Proc. Nat. Acad. Sci. 86:2766-2770. Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence difference between alleles of target sequences.

Association of a microsatellite marker, an SNP marker, or both, with a specific phenotype can be established using statistical analysis well known in the art. For example, if the frequency of a specific allele of a marker is significantly higher in a population with a genetic disorder, it can be concluded that the specific allele of the marker is indicative of the genetic disorder. In other words, a subject carrying the specific allele of the marker is likely to develop or display the genetic disorder. Linkage between a microsatellite marker and an SNP marker of a susceptible gene can be identified, e.g., based on linkage disequilibrium.

A diagnostic method of this invention involves providing a nucleic acid sample from a subject and identifying a microsatellite marker, an SNP marker, or both. The presence of the marker(s) indicates that the subject is suffering from or at risk for developing a genetic disorder. For example, the presence of a D5S2011 E marker, a CD14-F1826 T marker, or both, indicates that a subject is suffering from or at risk for developing an allergic disorder and is likely to have a serum IgE concentration of 1000 IU/ml or higher. A subject to be diagnosed can be, e.g., an individual displaying symptoms related to a disorder or having a family history of such a disorder. For the just-mentioned example, a subject can be a family member or a newborn with high IgE risk. The method of this invention can be used on its own or in conjunction with other procedures to diagnose a genetic disorder in an appropriate subject.

A kit can be provided for practicing the present invention. Such a kit contains one or more agents for detecting a microsatellite marker, an SNP marker, or both. The agent can be, for example, a hybridization probe or a pair of PCR primers, which may be conjugated to a detectable label. In some cases, an agent or agents can be packaged in a container with a label or an insert to indicate the intended uses of the agent(s), i.e., diagnosis of a genetic disorder.

The specific example below is to be construed as merely illustrative, and not limitative of the remainder of the disclosure in any way whatsoever. Without further elaboration, it is believed that one skilled in the art can, based on the description herein, utilize the present invention to its fullest extent. All publications recited herein are hereby incorporated by reference in their entirety.

Materials and Methods

(1) Clinical Sample Collection

Asthmatic patients and age-matched controls were divided by their serum IgE levels into two groups: high IgE group (serum IgE concentration ≧1000 IU/ml) and low IgE group (serum IgE concentration ≦200 IU/ml). A total of 200 samples were collected during 1988-2001 at National Cheng-Kung University Hospital, Tainan, Taiwan, a regional referral center for patients with asthma and other airway obstruction diseases. Patients who had symptomatic asthma without a current asthma exacerbation were referred to this hospital and were examined for a standardized, complete evaluation at the out-patient clinic. At the time of initial testing, all subjects with asthma symptoms were hyper-responsive to histamine (PC₂₀ forced expiratory volume in 1 s (FEV₁), 32 mg of histamine/ml, 30 s method), and were <16 years old.

(2) Clinical Evaluation

Pulmonary function was tested using standard methods that included spirometry before and after the administration of inhaled salbutamol (800 mg). Testing of bronchial responsiveness to histamine was performed using the method of De Vries et al. ((1962) Int Arch Allergy 20:93-101), which had been used to assess the initial participants during the period of 1962-75. The reactivity-testing protocol consisted of the subject inhaling increasing concentrations of histamine, for 30 s of tidal breathing, to a maximum dose of 32 mg of histamine/ml. The test was stopped if FEV₁ of the individual decreased 20%. Other evaluations included skin tests for responsiveness to 16 common allergens (intracutaneous testing in adults and prick testing in children), a differential blood count (including total eosinophil count), and measurements of total serum IgE, as well as IgE specific to house dust and mixed pollens. A positive skin test was defined as the presence of 1 reaction with a wheal diameter of 5 mm. Total serum IgE was measured using a solid-phase immunoassay (Pharmacia IgE EIA, Pharmacia Diagnostics).

(3) STR Genotyping

Genotyping was performed using ABI PRISM Linkage Mapping Sets MD-10 (400 markers). These markers are arranged in MD-10 sets to provide coverage of human genome at an average resolution of 10 cM. Each marker set includes a fluorescence labeled forward primer and a tailing reverse primer. Reverse primer tailing chemistry, placing the sequence GTTTCTT at the 5′-end of reverse primers, was used to promote the non-template directed nucleotide addition during amplification, which resulted in consistent allele calls and more precise data output. The PCR reaction contained 9.0 ul True Allele PCR Premix (including dNTPs, buffer, MgCl₂, and Tag DNA polymerase), 3.8 ul sterile de-ionized water, 1.0 ul primer pair (5 uM each primer), 1.2 ul genomic DNA (50 ng) and was set up in a 96-well microtiter plate. Amplification was carried out in 9700 PCR machines (ABI) with the following thermal reactions: one cycle at 95° C. for 12 min; 10 cycles of melting at 94° C. for 15 sec, annealing at 55° C. for 15 sec, and extending at 72° C. for 30 sec; 20 cycles of melting at 89° C. for 15 sec, annealing at 55° C. for 15 sec, and extending at 72° C. for 30 sec; and one cycle of final extension at 72° C. for 10 min.

After PCR, the reaction products were pooled with a panel of markers at a 1:1:2 ratio (FAM:VIC:NED) for a single capillary injection. 0.5 ul of pooled PCR product was mixed with 9 ul of a formamide:size standard mixture, which was prepared by mixing 50 ul of GeneScan-500 LIZ Size Standard with 900 ul of Hi-Di formamide. DNA dispensing and pooling of PCR products were performed with separate pipetting robots to ensure a fast and error-free liquid handing process. PCR pools were separated on ABI 3700 DNA Analyzers. The use of GeneScan 500 LIZ as the internal size standard assisted polymorphic fragment length calling and allowed more accurate allele calling and unambiguous comparison of data across various experimental conditions. Genotypes were scored using Genescan and Genotyper (ABI) software, and were checked independently by three individuals, without prior knowledge of the corresponding phenotypes.

Association of STR markers with the IgE level was identified using Monte-Carlo estimation (SAS10.0, SAS Inc.). Alleles of significant markers were tested for linkage disequilibrium by analysis of contingency tables. Significant alleles were then tested for risk factor by odd ratio.

(4) SNP Genotyping

DNA Fragment flanking the promoter region of the CD14 gene was amplified by four separate PCRs using four pairs of forward and reverse primers. The primers used are as follow:

| | |
|---|---|
| SEQ ID NO:1: | GTG CCA ACA GAT GAG GTT CAC, |
| SEQ ID NO:2: | CGC AGC GGA AAT CTT CAT C, |
| SEQ ID NO:3: | CTA GCT TCT AAG ACC CAC ACT TGG, |
| SEQ ID NO:4: | CTT TCA GAG AAC TCA GGC CAC TG, |
| SEQ ID NO:5: | ACA CCC ACC AGA GAA GGC TTA GG, |
| SEQ ID NO:6: | CCT ACC AGT AGC TGA GCA GGA ACC, |
| SEQ ID NO:7: | CTA GAC CTC AGC CAC AAC TCG, and |
| SEQ ID NO:8: | GGG AAG TGC ATA GGA GAG GAA A. |

The reaction mixture consisted of Tris-HCl 100 mM (pH 8.3), KCl 50 mM, MgCl₂ 2.5 mM, 40 ng genomic DNA, 0.2 mM dNTP, 0.25 μM forward primer and reverse primer, 5 U Taq DNA polymerase, and 0.05 U Pfu DNA polymerase in a total volume of 50 μl. Amplification was carried out on 9700 PCR machines (ABI) with four different thermal cycling parameters. For the primer pair of SEQ ID NO:1 and SEQ ID NO:2, the following thermal conditions were applied: one cycle at 94° C. for 4 min; 35 cycles of melting at 94° C. for 40 sec, annealing at 65° C. for 40 sec, and extending at 72° C. for 1 min 30 sec; and one cycle of final extension at 72° C. for 10 min. For primers of SEQ ID NOs:3-8, a touch-down program was used: an initial denaturing at 94° C. for 4 min; 10 cycles of melting at 94° C. for 40 sec, annealing at 65° C. for 40 sec, and extending at 72° C. for 1 min 30 sec; for the subsequent 25 cycles, the annealing temperature was decreased 0.25° C. per cycle; and one cycle of final extension at 72° C. for 10 min. The PCR products were purified by membrane ultra-filtration with MultiScreen PCR plate (Millipore) according to the manufacture's instructions.

Each amplified and purified reaction product was sequenced using the forward or reverse primer separately. The sequencing reaction was performed in a PCR machine with each reaction mixture consisting of 1-3 μl of the purified PCR product, Big Dye Terminator Ready-Reaction-Premix and 10 pmol of a sequencing primer. The reaction was subjected to 28 cycles at 94° C. for 30 sec, 52° C. for 30 sec, and 58° C. for 2 min. The reaction product was purified by ethanol precipitation, re-suspended in de-ionized formamide, and loaded on an ABI 3700 capillary sequencer.

The sequence data were analyzed using PolyPhred software to identify candidate SNPs associated with the IgE level. The candidate SNPs were manually checked to ensure the presence of true SNPs and alleles of each individual. Independent manual confirmations were performed for all sequence data and only those confirmed were subsequently subjected to statistical analysis.

Association between the IgE level and the CD14 single nucleotide polymorphism, including allele frequency and genotype frequency, was analyzed by χ² test or fishers' exact test.

Results

(1) Association of D5S2011 Markers With Serum IgE Concentration Among Asthma Patients

Twelve D5S2011 alleles (A-L) were identified among all asthma patients. Distribution of the alleles, genotypes, and serum IgE levels are summarized in Tables 1-5 below. The D5S2011 E allele and EE+EX genotypes were found to be associated with high IgE levels. In contrast, the D5S2011 J allele and JJ+JX genotypes were found to be associated with low IgE levels.

TABLE 1
D5S2011 alleles and associated serum IgE levels

| | | Patients with | Patients with | |
|---|---|---|---|---|
| Alleles | Size (bp) | high IgE levels | low IgE levels | P-value^{a} |
| | | 210 | 170 | 0.003 |
| A | 129 | 5 (2%) | 4 (2%) | |
| B | 133 | 0 (0%) | 1 (1%) | |
| C | 139 | 0 (0%) | 1 (1%) | |
| D | 141 | 3 (1%) | 8 (5%) | |
| E | 143 | 67 (32%) | 28 (16%) | |
| F | 145 | 61 (29%) | 45 (26%) | |
| G | 147 | 33 (16%) | 37 (22%) | |
| H | 149 | 13 (6%) | 12 (7%) | |
| I | 151 | 12 (6%) | 8 (5%) | |
| J | 153 | 7 (3%) | 18 (11%) | |
| K | 155 | 8 (4%) | 8 (5%) | |
| L | 157 | 1 (0%) | 0 (0%) | |
| Heterozygosity^{b} | | 0.779 | 0.832 | |

^{a}P-value: Monte Carlo exact test for differentiation between high IgE and low IgE groups of patients
^{b}Heterozygosity: 1 − sum (pi\hat{ }2)

TABLE 2
D5S2011 E allele and associated serum IgE levels

| | Patients with | Patients with |
|---|---|---|
| | high IgE levels | low IgE levels |
| D5S2011 E allele | 67 | 28 |
| Other D5S2011 alleles | 143 | 142 |

OR (Odds Ratio)=2.38, 95% CI (Confidence Interval)=(1.44, 3.91)

TABLE 3
D5S2011 EE + EX genotypes and associated serum IgE levels

| | Patients with | Patients with |
|---|---|---|
| | high IgE levels | low IgE levels |
| D5S2011 EE + EX genotypes | 56 | 24 |
| Other D5S2011 genotypes | 49 | 61 |

OR=2.90, 95% CI=(1.58, 5.34)

TABLE 4
D5S2011 J allele and associated serum IgE levels

| | Patients with | Patients with |
|---|---|---|
| | high IgE levels | low IgE levels |
| D5S2011 J allele | 7 | 18 |
| Other D5S2011 alleles | 203 | 152 |

OR=0.29, 95% CI=(0.12, 0.71)

TABLE 5
D5S2011 JJ + JX genotypes and associated serum IgE levels

| | Patients with | Patients with |
|---|---|---|
| | high IgE levels | low IgE levels |
| D5S2011 JJ + JX genotypes | 7 | 17 |
| Other D5S2011 genotypes | 98 | 68 |

OR=0.29, 95% CI=(0.11, 0.73)

(2) Association of CD14-F1826 T Marker With Serum IgE Concentration Among Asthma Patients

Distribution of CD14-F1826 alleles, genotypes, and serum IgE levels are summarized in Tables 6 and 7 below. The CD 14-F1826 T allele and TT+CT genotypes were found to be associated with high IgE levels.

TABLE 6
CD14-F1826 alleles and associated serum IgE levels

| | Patients with | Patients with |
|---|---|---|
| | high IgE levels | low IgE levels |
| CD14-F1826 T allele | 129 | 82 |
| CD14-F1826 C allele | 69 | 68 |

χ²=3.93, P value=0.0474, OR=1.55, 95% CI=(1.004, 2.394)

TABLE 7
CD14-F1826 genotypes and associated serum IgE levels

| | Patients with | Patients with |
|---|---|---|
| | high IgE levels | low IgE levels |
| CD14-F1826 TT + CT genotypes | 86 | 56 |
| CD14-F1826 CC genotype | 13 | 19 |

χ²=4.233, P value=0.0396, OR=2.245, 95% CI=(1.03, 4.90)

(3) Association of D5S2011 E Marker and CD14-F1826 T Marker With Serum IgE Concentration Among Asthma Patients

Unexpectedly, the combination of the D5S2011 E marker and the CD 14-F1826 T marker was found to be a more effective indicator for high serum IgE levels than each individual marker (Tables 8 and 9).

TABLE 8
D5S2011 and CD14-F1826 genotypes and associated serum IgE levels

| | Patients with | Patients with |
|---|---|---|
| | high IgE levels | low IgE levels |
| EE + EX/TT + CT | 46 | 15 |
| XX/TT + CT | 40 | 41 |
| EE + EX/CC | 6 | 7 |
| XX/CC | 7 | 12 |

χ²=14.117, P value ˜0.0027

TABLE 9
D5S2011 EE + EX and CD14-F1826 TT + CT
genotypes and associated serum IgE levels

| | Patients with | Patients with |
|---|---|---|
| | high IgE levels | low IgE levels |
| EE + EX/TT + CT | 46 | 15 |
| XX/CC | 7 | 12 |

OR=5.257, 95% CI=(1.75, 15.78)

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A method of developing a means for detecting a genetic disorder, the method comprising identifying a microsatellite marker and a single nucleotide polymorphism marker, wherein presence of both the microsatellite marker and the single nucleotide polymorphism marker in a subject indicates that the subject is suffering from or at risk for suffering from a genetic disorder.

2. A kit for detecting a genetic disorder, the kit comprising:
a first agent for detecting a microsatellite marker, and
a second agent for detecting a single nucleotide polymorphism marker,
wherein presence of both the microsatellite marker and the single nucleotide polymorphism marker in a subject indicates that the subject is suffering from or at risk for suffering from a genetic disorder.

3. A method of determining whether a subject is suffering from or at risk for developing a genetic disorder, the method comprising:
providing a nucleic acid sample from a subject, and
detecting a microsatellite marker and a single nucleotide polymorphism marker,
wherein detection of both the microsatellite marker and the single nucleotide polymorphism marker indicates that the subject is suffering from or at risk for suffering from a genetic disorder.

4. The method of claim 3, wherein the microsatellite marker is a D5S2011 E marker, the single nucleotide polymorphism marker is a CD14-F1826 T marker, and detection of both the D5S2011 E marker and the CD14-F1826 T marker indicates that the subject is suffering from or at risk for suffering from an allergic disorder and having or at risk for having a serum IgE concentration of 1000 IU/ml or higher.

5. The method of claim 4, wherein the allergic disorder is asthma.

6. A kit for detecting an allergic disorder, the kit comprising:
a first agent for detecting a D5S2011 E microsatellite marker, and
a second agent for detecting a CD14-F1826 T single nucleotide polymorphism marker,
wherein presence of both the D5S2011 E marker and the CD14-F1826 T marker in a subject indicates that the subject is suffering from or at risk for suffering from an allergic disorder and having or at risk for having a serum IgE concentration of 1000 IU/ml or higher.

7. The kit of claim 6, wherein the allergic disorder is asthma.

8. A method of subtyping an allergic disorder, the method comprising:
providing a nucleic acid sample from a subject suffering from or being at risk for suffering from an allergic disorder, and
detecting a D5S2011 E microsatellite marker,
wherein detection of the D5S2011 E marker indicates that the subject is having or at risk for having a serum IgE concentration of 1000 IU/ml or higher.

9. The method of claim 8, wherein the allergic disorder is asthma.

10. A packaged product comprising:
a container,
an agent for detecting a D5S2011 E microsatellite marker, and
a legend associated with the container and indicating that presence of the D5S2011 E marker in a subject indicates that the subject is suffering from or at risk for suffering from an allergic disorder and having or at risk for having a serum IgE concentration of 1000 IU/ml or higher.

11. The packaged product of claim 10, wherein the allergic disorder is asthma.

12. A method of subtyping an allergic disorder, the method comprising:
providing a nucleic acid sample from a subject suffering from or being at risk for suffering from an allergic disorder, and
detecting a D5S2011 J microsatellite marker,
wherein detection of the D5S2011 J marker indicates that the subject is having or at risk for having a serum IgE concentration of 200 IU/ml or lower.

13. The method of claim 12, wherein the allergic disorder is asthma.

14. A packaged product comprising:
a container,
an agent for detecting a D5S2011 J microsatellite marker, and
a legend associated with the container and indicating that presence of the D5S2011 J marker in a subject indicates that the subject is suffering from or at risk for suffering from an allergic disorder and having or at risk for having a serum IgE concentration of 200 IU/ml or lower.

15. The packaged product of claim 14, wherein the allergic disorder is asthma.

16. A method of subtyping an allergic disorder, the method comprising:
providing a nucleic acid sample from a subject suffering from or being at risk for suffering from an allergic disorder, and
detecting a CD14-F1826 T single nucleotide polymorphism marker, wherein detection of the CD14-F1826 T marker in a subject indicates that the subject is having or at risk for having a serum IgE concentration of 1000 IU/ml or higher.

17. The method of claim 16, wherein the allergic disorder is asthma.

18. A packaged product comprising:
a container,
an agent for detecting a CD14-F1826 T single nucleotide polymorphism marker, and
a legend associated with the container and indicating that presence of the CD14-F1826 T marker in a subject indicates that the subject is suffering from or at risk for suffering from an allergic disorder and having or at risk for having a serum IgE concentration of 1000 IU/ml or higher.

19. The packaged product of claim 18, wherein the allergic disorder is asthma.
